(19) United States
(12) Patent Application Publication
(10) Pub. No.: US 2020/0163913 A1
(43) Pub. Date: May 28, 2020

(54) CA-4 Antitumour Drug, Synthesis Method and Use Thereof

(71) Applicant: Shanghai Huali Biomedical Co., Ltd., Shanghai (CN)

(72) Inventors: Fanhong Wu, Shanghai (CN); Leilei Huang, Shanghai (CN); Jinwen Huang, Shanghai (CN); Yingzi Li, Shanghai (CN); Da Xie, Shanghai (CN); Meichen Hu, Shanghai (CN)

(21) Appl. No.: 16/638,355

(22) PCT Filed: Jul. 24, 2018
(86) PCT No.: PCT/CN2018/096836

§ 371 Date: Feb. 11, 2020

(30) Foreign Application Priority Data
Jul. 25, 2017 (CN) 201710610751.9
Aug. 11, 2017 (CN) 201710685120.3

(51) Int. Cl. A61K 31/18; A61P 35/04

(52) U.S. Cl. CPC A61K 31/18; A61P 35/04

(57) ABSTRACT

The invention discloses CA-4 antitumour drug, their synthetic methods and applications. The CA-4 antitumour drug are obtained by introducing an alkoxy group or a fluorine-containing alkoxy group at the 4′ position of the natural product Combretastatin and modified with a functional chemical group at its 3′ position. The CA-4 derivated anti-tumor drugs of the invention have inhibitory ability on two targets related to tubulin and arylsulfatase, and can be used for anti-tumor treatment. t,?

### CROSS-REFERENCE TO RELATED APPLICATION

This application claims priority from Chinese Patent Application No. 201710685120.3, filed on Aug. 11, 2017, in State Intellectual Property Office of P.R. China, the contents of which are hereby incorporated by reference in their entirety for all purposes.

### BACKGROUND OF THE INVENTION

### 1. Technical Field

The invention is related to the technical field of drug synthetic, in particular to the CA-4 antitumour drug, their synthetic methods and applications.

### 2. Description of Related Art

In today's world, cancer is one of the diseases with the highest prevalence and mortality in the world. It has a serious impact on human health and human life, and has become the second killer that threatens human health. In recent years, the research and development of anti-tumor drugs has made great progress in chemotherapy methods. Combretastatin-4 (CA-4) is a type of stilbene compound extracted and isolated from the trunk of the Combretum caffrum in South African bush willow bark. It has a strong anti-tumor effect, and its mechanism of action is related to the inhibition of tubulin polymerization. It is one of the most active tubulin inhibitors currently known. It is also related to the traditional tubulin inhibitors paclitaxel and vincristine. Unlike alkali, CA-4 has a selective destruction effect on tumor blood vessels, which makes tumors starve to death due to lack of nutrient supply. Its pharmacological action mechanism and structural modification work are also continuously deepened. Although CA-4 has good anti-tumor activity, CA-4 and its derivatives currently have many defects, such as poor water solubility, unstable cis structure, and inactive trans structure. Great obstacle. Therefore, a lot of research work has been done around the structural transformation of CA-4 analogs.

As shown in the above chemical formulas, in most cases, in the structural transformation of CA-4, the trimethoxyphenyl group of the A ring, which has been proved to be a functional group necessary for preservation, has been modified by modification on the A ring. Or lost, so the structural modification mainly focuses on the modification of the connecting bridge and the B ring, such as replacing the cis double bond with a rigid ring for conformational fixation, etc. The modification on the B ring was the first breakthrough, and Pettit and others designed and synthesized Introducing disodium phosphate at the hydroxyl position of ring B to obtain the phosphorylated disodium salt of CA-4, the prodrug of compritin disodium phosphate CA4P, is currently in the phase III clinical research phase. CA4P greatly improves the water-solubility and pharmacokinetic properties of CA-4. By using the characteristic that the concentration of phosphatase in proliferating vascular endothelial cells is higher than that of normal cells, CA4P is selectively activated in tumor vessels and targeted for release CA4P also exerts anti-vascular and anti-tumor effects. In addition, the activity of the product in which the 3-position hydroxyl group of the B ring is replaced with an amino group and further condensed with the amino acid is enhanced. Among them, the serine derivative has a better oral absorption effect, and it is currently entering the clinical II research stage.

Arylsulfatase or Arylsulphatase (ARS) is a family of proteins that catalyze the hydroysis of sulfates. There are currently four sulfatase in depth studies, namely lysosomal arylsulfatase (ARS-A and ARS-B), endoplasmic reticulum arylsulfatase (ARS-C) and extracellular arylsulfatase (HSulf-1). In the human body, it regulars the sulfate levels of many bio-molecules, this process is closely related to hormone regulation in cells, degradation of cell components, and regulation of signaling pathways. In recent years, a large number of studies have found that the arylsultatase level have a significant correlation with tumor development. The serum ARS activity of patients with various types of tumor is normal, but the enzyme activity in urine is significantly increased, such as the urine ARS activity in bladder cancer patients The average value can reach 40 times the normal value. The positive rate of ARS-A activity in the urine of leukemia patients is 100%, and ARS-B is 97.5%. Breast cancer HSulf-1 is down-regulated in mRNA levels, and angiogenesis in breast cancer Regulated by HSulf-1. Except for blood diseases, patients with colon cancer, gastric cancer, and bladder cancer had the highest sulfatase enzyme activity, followed by breast cancer, cervical cancer, prostate cancer, kidney cancer, and skin cancer patients. The detection of ARS activity in morning urine confirmed that in breast cancer, colorectal cancer, and urinary system cancer, the comprehensive activity of ARS was more specific for the detection of male and female samples, which were 96.2% and 95.3% [Chin J Heal Lab Tech, 2011, 21 (2): 429.], in breast and colorectal cancer samples, ARS positive rates were higher than CEA, CA199, CA72. 4. CA242, CA153 and other tumor markers [World Chinese Journal of Digestion, 2009, 17 (28): 2964], in the detection of breast cancer samples, the sensitivity of ARS activity was 57.0%, the specificity was 97.8%, and the accuracy was 69.9%. Therefore, drug design that takes advantage of the high arylsulfatase activity in cancer patients has become a hot spot in current anti-tumor drug research.

Sulfamate groups are an important class of pharmacophores that can be hydrolyzed by sulfatase in the body. Based on this principle, small molecule arylsulfatase inhibitors may be effective in cancer therapy by regulates the sulfate level of biological molecules, small molecule drugs arylsulfonate inhibitors Emate (marketed) and STX64 (clinical phase I) are the best examples. The B ring of CA-4 has a modifiable phenolic hydroxyl group. There have been many researches on CA-4 in the past, but there have been no reports of derivatives that introduce a sulfamate group at the 3-position of ring B. The introduction of sulfamate on CA-4 is also a kind of prodrug modification, it can be hydrolyzed by sulfatase to release CA-4. Therefore, the sulfamate of CA-4 may play as a multiple target anti-tumor agents.

### SUMMARY OF THE INVENTION

In order to overcome the shortcomings of the prior art, the object of the present invention is to provide the new anti-tumor drugs derivated from CA-4, their synthetic methods and applications.

The technical solution of the present invention is specifically described as follows.

The invention provides CA-4 antitumour drug, the structures of which are shown in Formula I:

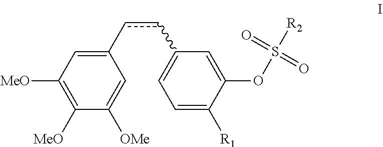

Where: “---” is a bond or does not exist

R₁ is OMe, OEt, OCF₂H or H; R₂ is NH₂, NHMe or N(CH₃)₂.

The invention also provides a method for synthesizing CA-4 antitumour drug. The specific steps are as follows:

(1) The compounds having the structure shown in Formula 02 are obtained by a compound having the structure shown in Formula 01 reacting with chlorotriphenylmethane.

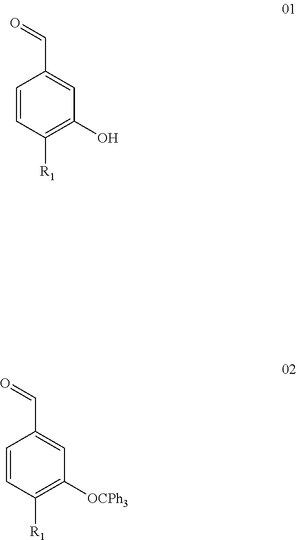

(2) The compounds with structures shown in Formulas 03 and 04 are prepared from Witting reaction of 3,4,5-trimethoxybenzyl triphenylphosphonium bromide and formula 02 using n-butyl lithium.

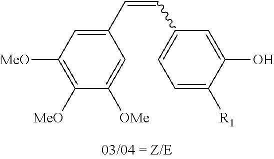

(3) The compound of the structure shown by Formula I are obtained by reacting the sulfamoyl chlorides with a compound of the structure shown by Formula 03 and Formula 04 under a basic condition.

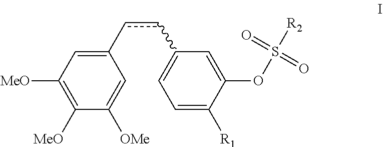

Where: “---” is a bond or does not exist.

R₁ is OMe, OEt, OCF₂H or H; R₂ is NH₂, NHMe or N(CH₃)₂.

The present invention also provides CA-4 antitumour drug, the structure of which is shown in Formula II:

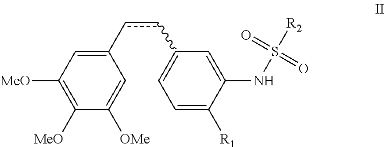

Where: “---” is a bond or does not exist

R₁ is OMe, OEt, OCF₂H or H; R₂ is NH₂, NHMe or N(CH₃)₂.

Further, the present invention is also CA-4 antitumour drug, the structure of which is shown by the general formula IV:

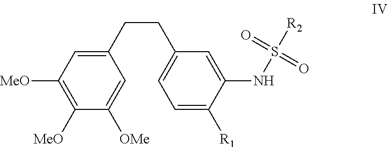

Where:

R₁ is OMe, OEt, OCF₂H or H; R₂ is NH₂, NHMe or N(CH₃)₂.

The invention also provides a method for synthesizing CA-4 antitumour drug. The specific steps are as follows:

(1) The compounds with structures shown in Formulas 06 and 07 are prepared from Witting reaction of 3,4,5-trimethoxybenzyl triphenylphosphonium bromide and formula 05 using n-butyl lithium

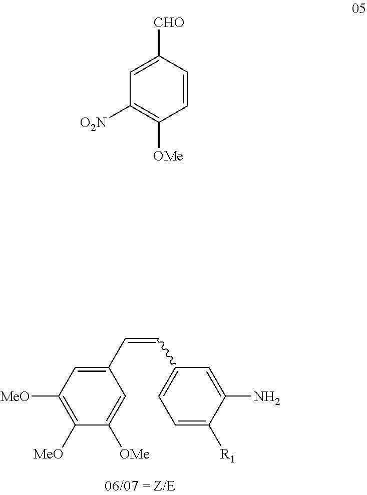

(2) The compounds of the general formula III are prepared by reacting the compounds of the formulas 06 and 07 with sulfamoyl chlorides under basic condition.

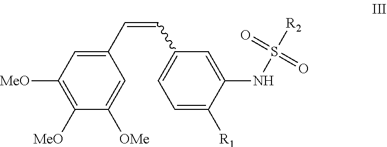

(3) The compounds with general formula IV are obtained from reduction of the compounds (formula III) using palladium-carbon catalyst.

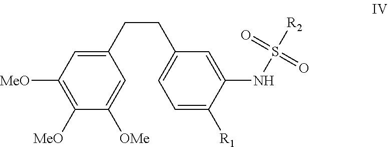

Where:

R₁ is OMe, OEt, OCF₂H or H; R₂ is NH₂, NHMe or N(CH₃)₂.

The invention also provides the applications of CA-4 antitumour drug in the preparation of a tubulin aggregation inhibitor.

The invention further provides the applications of CA-4 antitumour drug in the preparation of a medicament that acts as an anti-tumor vascular disrupting agent and has vascular targeting effect on various tumors. Anti-tumor vascular disrupting agent, applications in medicine with vascular targeting effect on various tumors. The various tumors include: lung cancer, non-small cell lung cancer, liver cancer, pancreatic cancer, gastric cancer, bone cancer, esophageal cancer, breast cancer, prostate cancer, testicular cancer, colon cancer, ovarian cancer, bladder cancer, cervix Cancer, melanoma, squamous cell carcinoma, basal cell carcinoma, adenocarcinoma, sweat adenocarcinoma, sebaceous adenocarcinoma, papillary carcinoma, papillary adenocarcinoma, cystic adenocarcinoma, cystic carcinoma, myeloid carcinoma, bronchial carcinoma, osteoma, Epithelial cancer, cholangiocarcinoma, choriocarcinoma, embryo cancer, seminoma, Wilms cancer, glioblastoma, astrocytoma, neuroblastoma, craniopharyngioma, ependymal tumor, Pineal tumor, hematopoietic tumor, vocal cord neuroma, meningioma, neuroblastoma, optic neuroblastoma, retinoblastoma, neurofibromas, fibrosarcoma, fibroblastoma, fibromas, fibroadenomas, Fibrochondromas, Fibrocystoma, Fibromyxoma, Fibroostoma, Fibromyxaroma, Fibropapilloma, Myxosarcoma, Myxocystoma, Myxochondroma, Myxochondrosarcoma, Myxochondroma Fibrosarcoma, Mucinous Adenoma Myxoblastoma, liposarcoma, lipoma, lipoadenoma, adipoma, lipochondroma, lipofibromas, lipohemangioma, myxolipomas, chondrosarcoma, chondroma, chondroma, chordoma, villus Membranous adenoma, chorionic epithelioma, chorionoma, osteosarcoma, osteoblastoma, osteochondrofibroma, osteochondrosarcoma, osteochondroma, osteocystoma, osteodenoma, osteofibroma, bone fiber Sarcoma, Angiosarcoma, Hemangioma, Angiolipoma, Angiochondromatoma, Hemangioblastoma, Angiokeratinoma, Angioglioma, Hemangioendothelioma, Angiofibroma, Hemangioma, Angiolipoma, Angiolymph Tumors, angiomyolipoma, angiomyolipoma, angiomyoma, angiomyxoma, vascular reticuloendothelioma, lymphangiosarcoma, lymphogranuloma, lymphangioma, lymphoma, lymphomyxoma, lymphosarcoma, Lymphangioma, lymphoma, lymphoepithelial tumor, lymphoblastoma, endothelioma, endothelioma, synovial tumor, synovial sarcoma, mesothelioma, connective tissue tumor, especially Tumor, leiomyoma, leiomyosarcoma, leiomyoma, leiomyosarcoma, rhabdomyosarcoma, rhabdomyosarcoma, rhabdomyosarcoma, acute lymphoblastic leukemia, acute myeloid leukemia, chronic disease cells, erythrocytosis, lymphoma, multiple bone marrow tumor.

Further, the present invention provides applications of CA-4 antitumour drug in the preparation of drugs for treating diseases caused by abnormal neovascularization. The diseases mainly include: rheumatoid arthritis, diabetic retinopathy, precocious retinopathy, retinal vein occlusion, psoriasis, rosacea, Kaposi's sarcoma, specific reactive keratitis, epidemic keratoconjunctivitis, neovascularity Glaucoma, bacterial ulcer, fungal ulcer, simple scar infection, shingles infection, protozoan infection, mycobacterial infection, polyarteritis, sarcoma, scleritis, flushing, dry mouth, arthritis, dry mouth Disease, systemic lupus erythematosus, AIDS syndrome, syphilis.

For the efficacy and safety evaluation of the above CA-4 antitumour drug, the positive controls are as follows:

### (Z)-3,4,5-trimethoxy-3′-hydroxy-4′-methoxystilbene (Abbreviated Code: CA-4)

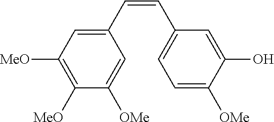 [SMILES: COc1ccc(/C=C\c2cc(OC)c(OC)c(OC)c2)cc1O]

### 3,4,5-trimethoxy-3′-hydroxy-4′-methoxydiphenylethane(Abbreviation: Erianin)

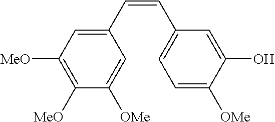 [SMILES: COc1ccc(/C=C\c2cc(OC)c(OC)c(OC)c2)cc1O]

For the efficacy and safety evaluation of the above CA-4 antitumour drug, the positive control to test sulfatase activity is as follows:

### 3-(sulfamoyloxy)-estr-1,3,5 (10)-triene-17-one (Abbreviated Code: EMATE)

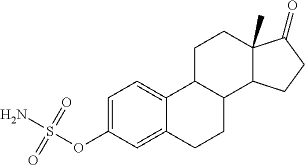 [SMILES: C[C@]12CCC3c4ccc(OS(N)(=O)=O)cc4CCC3C1CCC2=O]

The beneficial effects of the present invention are that the results of the pharmacodynamic and safety evaluation of the CA-4 antitumour drug of the present invention are summarized as follows:

Evaluation of the anti-tumor activity of tumor cells in vitro shows that compared with the positive control compounds CA-4 and Erianin, they have obvious anti-tumor activity on a variety of tumor cells cultured in vitro, and the anti-tumor activity is basically Equivalent, some compounds are significantly stronger than the positive control compounds. In particular, (Z)-3,4,5-trimethoxy-4′-ethoxy-3′-sulfamate stilbene has a significant effect on human lung cancer cell A549. Human colon cancer cells HTC-116, human cervical cancer cells Hela, human liver cancer cells HepG2, human gastric cancer cells MGC803, and human gastric cancer cells MKN45 have generally strong inhibitory activity (<0.5 μM).

Solid tumors depend on the vascular system for growth. Some rapidly proliferating tumor vascular endothelial cells rely on microtubules to maintain structural integrity due to the lack of intact myofilament structure. The rapid proliferation of proliferating human umbilical vein endothelial cells (HUVEC), It is more dependent on microtubules to keep the structure intact, so it is often used as an in vitro model of tumor vascular endothelial cells. Human umbilical vein endothelial cells (HUVEC) are used as objects to investigate the sulfamates of CA-4 antitumour drug. Derivatives have anti-tumor vascular properties and inhibit the proliferation of human umbilical vein endothelial cells, showing a class of very strong tubulin aggregation inhibitors, which are significantly stronger than the positive control compound CA-4 (IC50 value), indicating the sulfamates of CA-4 antitumour drug are a class of potentially strong tumor vessel targeting drugs.

Regarding the evaluation of in vitro sulfatase activity compared with EMATE, the results show that CA-4 antitumour drug have obvious stronger sulfatase inhibitory activity than the positive control compounds, such as (Z)-3,4,5-trimethoxystilbene-3′-sulfamate with strong inhibitory activity (<0.5 μM).

The anti-tumor drugs of the present invention can be an active target which can greatly improve its tumor blood vessel targeting activity compared with CA-4.

### BRIEF DESCRIPTION OF THE DRAWINGS

FIG. 1: Synthesis Route of (Z/E)-3,4,5-trimethoxy-4′-R₁-3′-sulfamate stilbene and 3,4,5-trimethoxy-4′-R₁-3′-sulfamate diphenylethane.

FIG. 2: Synthesis Route of (Z/E)-3,4,5-trimethoxy-4′-R₁-3′-aminosulfamate stilbene and 3,4,5-trimethoxy-4′-R₁-3′-aminosulfamate diphenylethane.

### DESCRIPTION OF THE PREFERRED EMBODIMENTS

In the following, a clear and complete description will be made in conjunction with the technical solutions of the embodiments of the present invention. Obviously, the described embodiments are only a part of the embodiments of the present invention, rather than all the embodiments. Based on the embodiments of the present invention, all other embodiments obtained by a person of ordinary skill in the art without creative efforts shall fall within the protection scope of the present invention.

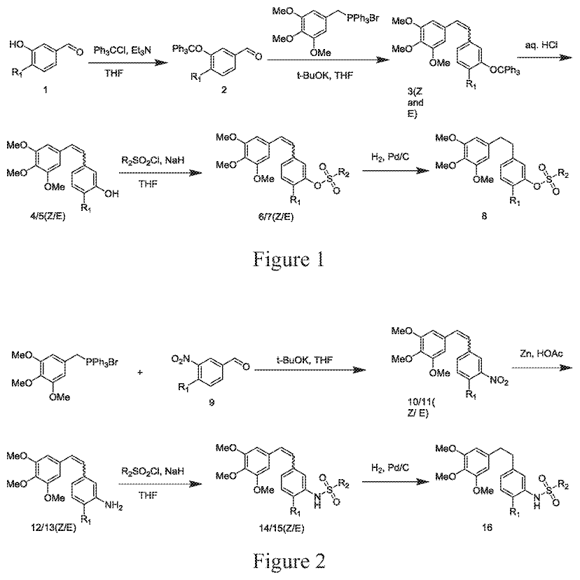

FIG. 1: Synthesis Route of (Z/E)-3,4,5-trimethoxy-4′-R₁-3′-sulfamate stilbene and 3,4,5-trimethoxy-4′-R₁-3′-sulfamate diphenylethane.

FIG. 2: Synthesis Route of (Z/E)-3,4,5-trimethoxy-4′-R₁-3′-aminosulfamate stilbene and 3,4,5-trimethoxy-4′-R₁-3′-aminosulfamate diphenylethane.

### Example 1: Synthesis of 3-triphenylmethoxy-4-methoxybenzaldehyde

hydroxy-4-methoxybenzaldehyde (8 g, 52.58 mmol), trityl chloride (16.8 g, 60.47 mmol), triethylamine (17.4 g, 17.23 mmol), and dry tetrahydrofuran (30 ml) are added to a 100 ml flask. The temperature is raised to reflux and the reaction is carried out for 5 hours While TLC tracking is implemented, an appropriate amount of water is added to the reaction system for stopping reaction. Ethyl acetate/n-heptane (1:1) are added, a light yellow granular solid is formed. The filtered cake is washed with distilled water, and dried under vacuum to afford 3-triphenylmethoxy-4-methoxybenzaldehyde (15.8 g, 76.7%).

### Example 2: Synthesis of (Z/E)-3,4,5-trimethoxy-4′-methoxy-3′-hydroxystilbene

Under nitrogen protection, a 250 mL three-necked flask is charged with (3,4,5-trimethoxybenzyl)triphenylphosphonium bromide (13.3 g, 25.37 mmol) and dried tetrahydrofuran (30 ml). The reactant is cooled to −78° C., and n-butyl lithium solution (15 ml) is added dropwise slowly. The system is stirred for 1 hour. A solution of 3-triphenylmethoxy-4-methoxybenzaldehyde (10 g, 25.37 mmol) in tetrahydrofuran (20 ml) is added slowly, and the temperature is raised to room temperature. The reaction is detected by TLC. A saturated brine is added, and the aqueous layer is separated. The organic phase is wished with a saturated brine, dried over anhydrous sodium sulfate, and concentrated. The residual solution is dissolved in toluene, and concentrated hydrochloric acid is added, followed by reacting at room temperature for 3 hours. The reaction is detected by TLC. An appropriate amount of water is added, extracted with ethyl acetate, dried over anhydrous sodium sulfate, and concentrated. (Z/E)-3,4,5-trimethoxy-4′-methoxy-3′-hydroxystilbene 6.4 g, (Z/E=3/1) is obtained by column chromatography eluting with petroleum ether/ethyl acetate (3/1). The yield is 76.4%.

### Example 3: Synthesis of (Z/E)-3,4,5-trimethoxy-4′-methoxy-3′-sulfamate stilbene

Under N₂ protection, NaH (0.15 g, 6.32 mmol) is added to a three-necked flask. A DMF solution (10 ml) of (Z/E)-3,4,5-trimethoxy-4′-methoxy-3′-hydroxystilbene (1 g, 3.16 mmol) is added slowly dropwise at 0° C. to the flask, and stirred at this temperature for 1 h. A solution of sulfamoyl chloride (0.82 g, 6.32 mmol) in DMF (2 ml) is added slowly to the reaction system. The reaction keeps going at room temperature overnight. After the reaction is completed, it is quenched with water and extracted with ethyl acetate. The organic phases are combined, washed with saturated aqueous sodium chloride solution, dried over anhydrous sodium sulfate, filtered, and concentrated. A yellow solid (0.72 g, 72%) are obtained by column chromatography eluting with petroleum ether/ethyl acetate (3/1).

### (Z)-3,4,5-trimethoxy-4′-methoxy-3′-sulfamate stilbene (A1-1)

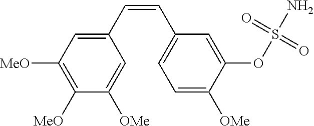 [SMILES: COc1ccc(/C=C\c2cc(OC)c(OC)c(OC)c2)cc1OS(N)(=O)=O]

¹H NMR (500 MHz, CDCl₃): δ 7.21 (s, 1H), 7.15 (d, J=8.5 Hz, 1H), 6.85 (d, J=8.5 Hz, 1H), 6.48 (d, J=12.1 Hz, 1H), 6.43 (d, J=12.1 Hz, 3H), 5.29 (s, 2H), 3.82 (d, J=13.8 Hz, 6H), 3.68 (s, 6H); HRMS-ESI (m/z) calculated for C₁₈H₂₁NO₇S [M+Na]⁺: 418.09364; Found: 418.09354, Melting point: 147.8-150.4° C.

### (E)-3,4,5-trimethoxy-4′-methoxy-3′-sulfamate stilbene (A1-2)

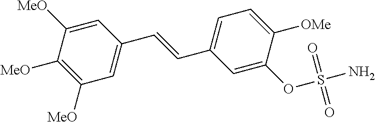 [SMILES: COc1ccc(/C=C/c2cc(OC)c(OC)c(OC)c2)cc1OS(N)(=O)=O]

¹H NMR (500 MHz, DMSO): δ 7.95 (s, 2H), 7.54 (s, 1H), 7.48 (d, J=8.5 Hz, 1H), 7.19 (d, J=16.0 Hz, 1H), 7.17 (s, 1H), 7.14 (d, J=16.0 Hz, 1H), 6.91 (s, 2H), 3.83 (s, 9H), 3.67 (s, 3H); HRMS-ESI (m/z) calculated for C₁₈H₂₁NO₇S [M+Na]⁺: 418.0936, Found: 418.0935.Melting point: 166.8-171.3° C.

### Example 4: Synthesis of 3,4,5-trimethoxy-4′-methoxy-3′-sulfamate diphenylethane (A1-3)

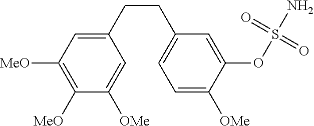 [SMILES: COc1ccc(CCc2cc(OC)c(OC)c(OC)c2)cc1OS(N)(=O)=O]

(Z/E) 3,4,5-trimethoxy-4′-methoxy-3′-sulfamate stilbene (0.3 g, 0.76 mmol) is dissolved in absolute ethanol (6 ml), the solution is added with 10% palladium on carbon (0.05 g), and hydrogen is feed, react at room temperature for 5 h. After the reaction is completed, it is filtered and concentrated to afford a colorless solid (0.22 g,73.3%). Melting point: 131.1-132.2° C. ¹H NMR (500 MHz, CDCl₃): δ 7.15 (s, 1H), 7.02 (d, J=10.0 Hz, 1H), 6.88 (d, J=8.4 Hz, 1H), 6.33 (s, 2H), 5.28 (s, 2H), 3.83 (s, 3H), 3.80 (s, 9H), 2.82 (dd, J=8.0, 3.8 Hz, 4H); HRMS-ESI (m/z) calculated for C₁₈H₁₉NO₇S [M+Na]⁺: 420.10929, Found: 420.10951, Melting point: 131.1-132.2° C.

### Example 5: Synthesis of (Z)-3,4,5-trimethoxy-4′-methoxy-3′-N-methylsulfamate stilbene (A1-4)

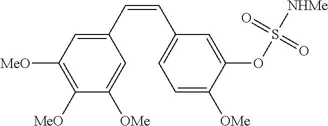 [SMILES: CNS(=O)(=O)Oc1cc(/C=C\c2cc(OC)c(OC)c(OC)c2)ccc1OC]

Under N₂ protection, NaH (0.08 g, 3.16 mmol) is added to a three-necked flask, and (Z)-3,4,5-trimethoxy-4′-methoxy-3′-hydroxystilbene is slowly added dropwise at 0° C. DMF solution (10 ml) of styrene (0.5 g, 1.58 mmol), reacted at this temperature for 1 h, and then a DMF solution (2 ml) of methylsulfamoyl chloride (0.41 g, 3.16 mmol) is added slowly to the reaction system. The reaction temperature is raised to room temperature and the reaction reacts overnight. After the reaction is completed, the reaction mixture is quenched with water, wished with saturated brine, and extracted with EA. The organic phases are combined, dried over anhydrous Na₂SO₄, filtered. A color solid is obtained by column chromatography eluting with petroleum ether/ethyl acetate (3/1). The yield is 66.2%. ¹H NMR (500 MHz, CDCl₃): δ 7.16 (s, 1H), 6.87 (d, J=9.0 Hz, 1H), 6.51 (d, J=12.0 Hz, 1H), 6.47 (d, J=12.0 Hz, 2H), 3.85 (s, 3H), 3.81 (s, 3H), 3.71 (s, 6H), 2.82 (d, J=5.2 Hz, 3H), 1.64 (s, 1H); HRMS-ESI (m/z) calculated for C₁₉H₂₃NO₇S [M+Na]⁺: 409.11952, Found: 409.11231; Melting point: 118.4-121.6° C.

### Example 6: Synthesis of (Z)-3,4,5-trimethoxy-4′-methoxy-3′-N,N-dimethylsulfamate stilbene (A1-5)

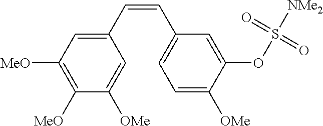 [SMILES: COc1ccc(/C=C\c2cc(OC)c(OC)c(OC)c2)cc1OS(=O)(=O)N(C)C]

Under N₂ protection, a three-necked flask is charged with NaH (0.08 g, 3.16 mmol), a DMF solution (10 ml) of (Z)-3,4,5-trimethoxy-4′-methoxy-3′-hydroxystilbene (0.5 g, 1.58 mmol) are added slowly dropwise at 0° C. The mixture reacts at this temperature for 1 h. A DMF solution (2 ml) of dimethyl sulfamoyl chloride (0.45 g, 3.16 mmol) is added slowly dropwise to the reaction system. The reaction temperature is raised to room temperature, and the reactant reacts overnight. After the reaction is completed, it is quenched with water and extracted with ethyl acetate. The organic phases are combined, washed with a saturated brine, dried over anhydrous Na₂SO₄ and filtered. A color solid is obtained by column chromatography eluting with petroleum ether/ethyl acetate (3/1). The yield is 76.1%. ¹H NMR (500 MHz, CDCl3): δ 7.21 (s, 1H), 7.10 (s, 1H), 6.82 (d, J=7.8 Hz, 1H), 6.42 (d, J=12.0 Hz, 1H), 6.44 (s, 1H), 6.46 (d, J=12.0 Hz, 1H), 6.47 (s, 1H), 3.85 (s, 3H), 3.82 (s, 3H), 3.67 (s, 3H), 3.64 (s, 3H), 2.88 (s, 3H), 2.86 (s, 3H); HRMS-ESI (m/z) calculated for C₂₀H₂₅NO₇S [M+Na]⁺: 446.13517, Found: 423.13541, Melting point: 102.7-105.6° C.

### Example 7: Synthesis of 3-triphenylmethoxy-4-ethoxybenzaldehyde

According to Example 1, 3-hydroxy-4-ethoxybenzaldehyde is used instead of 3-hydroxy-4-methoxybenzaldehyde with a yield of 78.6%.

### Example 8: Synthesis of (Z/E)-3,4,5-trimethoxy-4′-ethoxy-3′-hydroxystilbene

According to Example 2, 3-triphenylmethoxy-4-ethoxybenzaldehyde is used instead of 3-triphenylmethoxy-4-methoxybenzaldehyde with a yield of 68.5%.

### Example 9: Synthesis of (Z)-3,4,5-trimethoxy-4′-ethoxy-3′-sulfamate stilbene (A2-1)

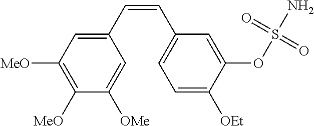 [SMILES: CCOc1ccc(/C=C\c2cc(OC)c(OC)c(OC)c2)cc1OS(N)(=O)=O]

According to Example 3, (Z)-3,4,5-trimethoxy-4′-ethoxy-3′-hydroxystilbene is reacted with sulfamoyl chloride to afford a pale yellow solid (0.47 g, 75.8%). ¹H NMR (500 MHz, CDCl₃): δ 7.21 (s, 1H), 7.14 (d, J=8.6 Hz, 1H), 6.86 (d, J=8.5 Hz, 1H), 6.49 (d, J=12.0 Hz, 1H), 6.44 (d, J=12.0 Hz, 3H), 5.15 (s, 2H), 4.10 (q, J=6.9 Hz, 2H), 3.82 (s, 3H), 3.69 (s, 6H), 1.43 (t, J=6.9 Hz, 3H); HRMS-ESI (m/z) calculated for C₁₉H₂₃NO₇S [M+Na]⁺: 432.10929, Found:432.10886; Melting point: 152.7-155.4° C.

### Example 10: Synthesis of (E)-3,4,5-trimethoxy-4′-ethoxy-3′-sulfamate stilbene (A2-2)

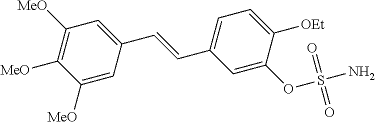 [SMILES: CCOc1ccc(/C=C/c2cc(OC)c(OC)c(OC)c2)cc1OS(N)(=O)=O]

According to Example 3, (E)-3,4,5-trimethoxy-4′-ethoxy-3′-hydroxystilbene is reacted with sulfamoyl chloride to afford a light yellow solid (0.47 g, 75.8%). ¹H NMR (500 MHz, CDCl₃): δ 7.18 (s, 1H), 7.11 (d, J=8.6 Hz, 1H), 6.96 (d, J=8.5 Hz, 1H), 6.63 (d, J=16.0 Hz, 1H), 6.58 (d, J=16.0 Hz, 3H), 5.18 (s, 2H), 4.21 (q, J=6.9 Hz, 2H), 3.93 (s, 3H), 3.71 (s, 6H), 1.48 (t, J=6.9 Hz, 3H); HRMS-ESI (m/z) calculated for C₁₉H₂₃NO₇S [M+Na]⁺: 432.10929, Found:432.10901; Melting point: 172.3-174.4° C.

### Example 11: Synthesis of 3,4,5-trimethoxy-4′-ethoxy-3′-sulfamate diphenylethane (A2-3)

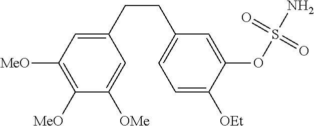 [SMILES: CCOc1ccc(CCc2cc(OC)c(OC)c(OC)c2)cc1OS(N)(=O)=O]

According to Example 4, (Z)-3,4,5-trimethoxy-4′-ethoxy-3′-sulfamate stilbene is used instead of (Z)-3,4,5-trimethoxy-4′-methoxy-3′-sulfamate stilbene, to afford a colorless solid (0.13 g, 68.4%). ¹H NMR (500 MHz, DMSO): δ 7.87 (s, 2H), 7.29 (d, J=7.2 Hz, 1H), 7.10 (dd, 1H), 7.04 (d, J=8.4 Hz, 1H), 6.54 (s, 2H), 4.04 (q, J=6.9 Hz, 2H), 3.74 (s, 6H), 3.61 (s, 3H), 2.80 (d, J=5.7 Hz, 4H), 1.32 (t, J=7.0 Hz, 3H); HRMS-ESI (m/z) calculated for C₁₉H₂₅NO₇S [M+Na]⁺: 434.12494, Found:434.12474; Melting point: 122.3-126.7° C.

### Example 12: Synthesis of 3-triphenylmethoxy-4-difluoromethoxybenzaldehyde

According to Example 1, 3-hydroxy-4-difluoromethoxybenzaldehyde is used instead of 3-hydroxy-4-methoxybenzaldehyde, and the yield is 72.3%.

### Example 13: Synthesis of (Z/E)-3,4,5-trimethoxy-4′-difluoromethoxy-3′-hydroxy stilbene

According to Example 2, 4-(difluoromethoxy)-3-(trityloxy)benzaldehyde is used instead of 3-triphenylmethoxy-4-methoxybenzaldehyde, and the yield is 63.8%.

### Example 14: Synthesis of (Z)-3,4,5-trimethoxy-4′-difluoromethoxy-3′-sulfamate stilbene (A3-1)

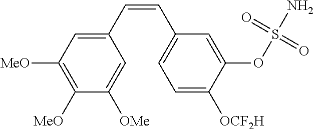

According to Example 3, (Z)-5-(3,4,5-trimethoxystyryl)-2′-(difluoromethoxy) phenol is used instead of (Z/E)-5-(3,4,5-trimethoxystyryl)-2-(difluoromethoxy) phenol, a pale yellow solid is obtained (0.29 g, 78.4%). ¹H NMR (500 MHz, DMSO): δ 8.50 (s, 2H), 7.05 (d, J=8.2 Hz, 1H), 7.01 (t, J=75, 1H), 6.92 (s, 1H), 6.72 (dd, J=8.2, 1.4 Hz, 1H), 6.54 (s, 2H), 6.52 (d, J=12.0 Hz, 1H), 6.49 (d, J=12.0 Hz, 1H), 3.64 (s, 3H), 3.58 (s, 6H); 19F NMR (470 MHz, DMSO) δ −81.38 (s); HRMS-ESI (m/z) calculated for C₁₈H₁₉F₂NO₇S [M+Na]⁺: 454.07480, Found: 454.07374; Melting point: 161.6-162.9° C.

### Example 15: Synthesis of (E)-3,4,5-trimethoxy-4′-difluoromethoxy-3′-sulfamate stilbene (A3-2)

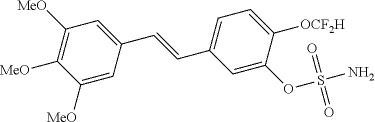

According to Example 3, (E)-5-(3,4,5-trimethoxystyryl)-2′-(difluoromethoxy)-phenol is used instead of (Z/E)-5-(3,4,5-trimethoxystyryl)-2′-(difluoromethoxy) phenol, a pale yellow solid is obtained (0.29 g, 78.4%). ¹H NMR (500 MHz, DMSO): δ 8.27 (s, 2H), 7.68 (s, 1H), 7.57 (d, J=8.4 Hz, 1H), 7.35 (d, J=8.4 Hz, 1H), 7.27 (d, J=16.0 Hz, 1H), 7.18 (d, J=16.0 Hz, 1H), 7.09 (t, J=75, 1H), 6.95 (s, 2H), 3.84 (s, 6H), 3.68 (s, 3H); ¹⁹F NMR (470 MHz, DMSO) δ −81.33 (s); HRMS-ESI (m/z) calculated for C₁₈H₁₉F₂NO₇S [M+Na]⁺: 454.07480, Found: 454.07477; Melting point: 145.5-148.6° C.

### Example 16: Synthesis of 3,4,5-trimethoxy-4′-ethoxy-3′-sulfamate diphenylethane (A3-3)

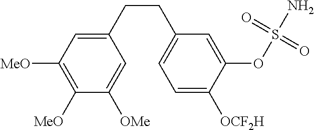

According to Example 4, 5-(3,4,5-trimethoxystyryl)-2′-(difluoromethoxy)phenyl-sulfamate is hydrogenated using 10% Pd—C catalyst to afford of a colorless solid(0.08 g, 61.7%). ¹H NMR (500 MHz, DMSO): δ 8.17 (s, 2H), 7.38 (d, J=7.0 Hz, 1H), 7.22 (s, 2H), 7.15 (t, J=75 Hz, 1H), 6.55 (s, 2H), 3.74 (s, 3H), 3.61 (s, 6H), 2.94-2.87 (m, 1H), 2.84-2.78 (m, 1H); ¹⁹F NMR (470 MHz, DMSO): δ −81.36 (s); HRMS-ESI (m/z) calculated for C₁₈H₂₁F₂NO₇S [M+Na]⁺: 456.09045, Found:456.08990; Melting point: 142.5-145.8° C.

### Example 17: Synthesis of 3-triphenylmethoxybenzaldehyde

According to Example 1.1,3-hydroxy-4-methoxybenzaldehyde is replaced with 3-hydroxybenzaldehyde, the yield is 70.8%.

### Example 18: Synthesis of (Z) and (E)-3,4,5-trimethoxy-3′-hydroxystilbene

According to Example 2, 3-triphenylmethoxybenzaldehyde is used instead of 3-triphenylmethoxy-4-methoxybenzaldehyde, and the yield is 66.7%.

### Example 19: Synthesis of (Z)-3,4,5-trimethoxy-3′-sulfamate stilbene (A4-1)

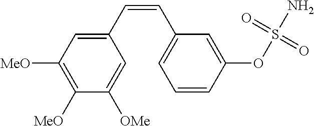 [SMILES: COc1cc(/C=C\c2cccc(OS(N)(=O)=O)c2)cc(OC)c1OC]

According to Example 3, (Z)-3,4,5-trimethoxy-3′-hydroxystilbene is reacted with sulfamoyl chloride to afford the title compound of a pale yellow solid (0.32 g, 76.5%). ¹H NMR (500 MHz, CDCl₃): δ 7.28 (s, 1H), 7.20 (d, J=7.6 Hz, 1H), 7.15 (s, 1H), 7.12 (d, J=8.0 Hz, 1H), 6.55 (d, J=12.0 Hz, 1H), 6.52 (d, J=12.0 Hz, 1H), 6.40 (s, 2H), 5.36 (s, 2H), 3.77 (s, 3H), 3.63 (s, 6H); HRMS-ESI (m/z) calculated for C₁₇H₁₉NO₆S [M+Na]⁺: 388.08253, Found:388.08247, [M+H]+: 366.10058, Found:366.10608; Melting point: 107.1-110.6° C.

### Example 20: Synthesis of (E)-3,4,5-trimethoxy-3′-sulfamate stilbene (A4-2)

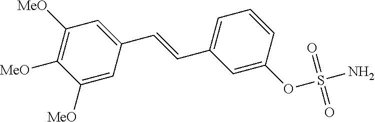 [SMILES: COc1cc(/C=C/c2cccc(OS(N)(=O)=O)c2)cc(OC)c1OC]

According to Example 3, (E)-3,4,5-trimethoxy-3′-hydroxystilbene is reacted with sulfamoyl chloride to afford the title compound of a pale yellow solid (0.32 g, 76.5%). ¹H NMR (500 MHz, CDCl₃): δ 7.43 (s, 1H), 7.40 (s, 1H), 7.36 (s, 1H), 7.20 (d, J=7.5 Hz, 1H), 7.00 (d, J=16.0 Hz, 1H), 6.91 (d, J=16.0 Hz, 1H), 6.69 (s, 2H), 5.28 (s, 2H), 3.87 (s, 6H), 3.85 (s, 3H); HRMS-ESI (m/z) calculated for C₁₈H₂₁F₂NO₇S [M+Na]⁺: 388.08253, Found:388.08341; Melting point: 136.3-139.7° C.

### Example 21: Synthesis of 3,4,5-trimethoxy-3′-sulfamate diphenylethane (A4-3)

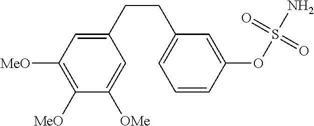 [SMILES: COc1cc(CCc2cccc(OS(N)(=O)=O)c2)cc(OC)c1OC]

According to Example 4, 3,4,5-trimethoxy-3′-hydroxy diphenylethane is hydrogenated using 10% Pd—C catalyst to afford of a colorless solid (0.1 g, 71.3%). ¹H NMR (500 MHz, CDCl₃): δ 7.29 (s, 1H), 7.11 (s, 1H), 7.06 (s, 1H), 6.30 (s, 2H), 5.42 (s, 2H), 3.77 (d, J=3.6 Hz, 9H), 2.87 (d, J=8.0 Hz, 2H), 2.83 (d, J=8.2 Hz, 2H); HRMS-ESI (m/z) calculated for C₁₇H₂₁NO₆S [M+Na]⁺: 390.09879, Found:390.09876; Melting point: 108.4-120.8° C.

### Example 22: (Z/E)-3,4,5-trimethoxy-4′-methoxy-3′-aminostilbene

Under nitrogen protection, a 250 ml three-necked flask is charged with (3,4,5-trimethoxybenzyl)triphenylphosphonium bromide (17.3 g, 33.12 mmol), dried tetrahydrogenfuran (20 ml), cooled to −78° C. A solution of n-butyl lithium (24 ml) is added dropwise slowly into the system, followed by stirring for 1 hour. A solution of 3-nitro-4-methoxybenzaldehyde (6 g, 33.12 mmol) in tetrahydrogenfuran (12 ml) is slowly added, and the temperature is raised to room temperature. The reaction is detected by TLC, saturated brine is added, and the aqueous layer is separated. The organic phase is washed with saturated brine, dried over anhydrous sodium sulfate, and concentrated to obtain a crude product.

The crude product is dissolved with excess acetic acid, 11 equivalents of zinc powder is added, and the reaction is processed in room temperature for 5 hours. After the reaction completed, water is added. The mixture is extracted with ethyl acetate, dried over anhydrous sodium sulfate, and concentrated. 3.1 g of (Z/E)-3,4,5-trimethoxy-4′-methoxy-3′-aminostilbene (Z/E=3/1) is obtained by column chromatography eluting with petroleum ether/ethyl acetate (3/1). The yield is 69.9%.

### Example 23: Synthesis of (Z)-3,4,5-trimethoxy-4′-methoxy-3′-aminosulfamate stilbene (A5-1)

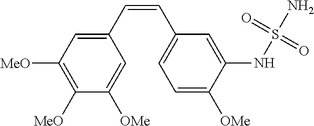 [SMILES: COc1ccc(/C=C\c2cc(OC)c(OC)c(OC)c2)cc1NS(N)(=O)=O]

According to Example 3, (Z)-3,4,5-trimethoxy-4′-methoxy-3′-aminostilbene is reacted with sulfamoyl chloride to afford the title compound of a brown solid (0.56 g, 78.2%). ¹H NMR (500 MHz, CDCl₃): δ 7.38 (d, J=1.7 Hz, 1H), 7.01 (dd, J=8.4, 1.8 Hz, 1H), 6.95 (s, 1H), 6.77 (d, J=8.4 Hz, 1H), 6.49 (d, J=4.2 Hz, 4H), 4.75 (s, 2H), 3.82 (d, J=2.4 Hz, 6H), 3.71 (s, 6H); HRMS-ESI (m/z) calculated for C₁₈H₂₂N₂O₆S [M+Na]⁺: 417.10966, Found: 417.10957; Melting point: 143.3-146.7° C.

### Example 24: Synthesis of (E)-3,4,5-trimethoxy-4′-methoxy-3′-aminosulfamate stilbene (A5-2)

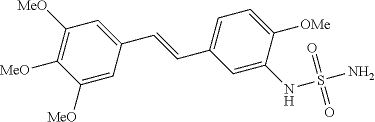 [SMILES: COc1ccc(/C=C/c2cc(OC)c(OC)c(OC)c2)cc1NS(N)(=O)=O]

According to Example 3, (E)-3,4,5-trimethoxy-4′-methoxy-3′-aminostilbene is reacted with sulfamoyl chloride to afford the title compound as of a brown solid (0.56 g, 78.2%). ¹H NMR (500 MHz, CDCl₃): δ 7.70 (s, 1H), 7.23 (d, J=7.7 Hz, 1H), 7.02 (s, 1H), 6.94 (s, 2H), 6.88 (d, J=8.0 Hz, 1H), 6.71 (s, 2H), 4.87 (s, 2H), 3.89 (d, J=11.0 Hz, 9H), 3.85 (s, 3H); HRMS-ESI (m/z) calculated for C₁₈H₂₂N₂O₆S [M+Na]⁺: 417.10966, Found:417.10992; Melting point: 157.9-161.1° C.

### Example 25: Synthesis of 3,4,5-trimethoxy-4′-methoxy-3′-aminosulfamate diphenylethane (A5-3)

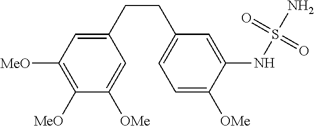 [SMILES: COc1ccc(CCc2cc(OC)c(OC)c(OC)c2)cc1NS(N)(=O)=O]

According to Example 4, 3,4,5-trimethoxy-4′-methoxy-3′-aminosulfamate stilbene is hydrogenated using 10% Pd—C catalyst to afford of a colorless solid (0.3 g, 68.7%). ¹H NMR (500 MHz, CDCl₃): δ 7.32 (s, 1H), 7.03 (s, 1H), 6.89 (d, J=8.1 Hz, 1H), 6.79 (d, J=8.3 Hz, 1H), 6.36 (s, 2H), 5.02 (s, 2H), 3.79 (d, J=6.9 Hz, 12H), 2.82 (d, J=3.4 Hz, 4H); HRMS-ESI (m/z) calculated for C₁₈H₂₄N₂O₆S [M+Na]⁺: 419.12531, Found:419.12514; Melting point: 122.5-126.5° C.

### Example 26 CCK-8 Method to Test the Anti-Tumor Activity of Compounds on Various Tumor Cells

### 1. Test Method

Take more than 90% of live cells for experiments. Cell proliferation inhibition test uses EnoGeneCell™ Counting Kit-8 (CCK-8) cell viability detection kit. Cells are digested and counted to make a cell suspension with a concentration of 1×105 cells/mL, 100 μL of cell suspension (1×104 cells per well) was added to each well of a 96-well plate; Incubate in a 5% CO2 incubator for 24 hours; add 100 μL of the corresponding drug-containing culture medium to each well, and set a negative control group, a vehicle control group, and a positive control group, with 5 replicates in each group; place the 96-well plate at 37° C. After incubation in a 5% CO2 incubator for 72 h; add 10 μL of CCK-8 solution to each well, incubate the culture plate in the incubator for 4 hours, measure the OD value at 450 nm with a microplate reader, calculate the target compound and Inhibition rates of Erianin and CA-4 positive drugs on human lung cancer cell A549, human colon cancer cell HCT-116, human cervical cancer cell HeLa, human liver cancer cell HepG2, human gastric cancer cell MGC-803, human gastric cancer cell MKN45 and other cells and IC₅₀ value.

### 2. Test Results

The experimental results show that the target compounds and the two positive controls of Erianin and CA-4 are positive for human lung cancer cell A549, human colon cancer cell HCT-116, human cervical cancer cell HeLa, human liver cancer cell HepG2, human gastric cancer cell MGC-803, human gastric cancer, cells MKN45 and other cells have different degrees of activity to inhibit cell proliferation. (Z)-3,4,5-trimethoxy-4′-methoxy-3′-aminosulfamatestilbene and (Z)-3,4,5-trimethoxy-4′-ethoxy-3′-aminosulfamatestilbene have significant cytotoxic activities on human lung cancer cell A549, and the IC50 values are below 0.5 μM. In addition, the two compounds also have significant cytotoxic activity on human colon cancer cells HCT-116 and human cervical cancer cells HeLa, and their IC50 values are 0.2216 μM and 0.446 μM; 0.3399 μM and 0.4799 μM. (Z)-3,4,5-trimethoxy-4′-methoxy-3′-aminosulfamatestilbene, (E)-3,4,5-trimethoxy-4′-methoxy-3′-aminosulfamatestilbene and (Z)-3,4,5-trimethoxy-4′-ethoxy-3′-aminosulfamatestilbene have significant cytotoxic activity to humans hepatocellular carcinoma HepG2, and the IC50 value are below 1 μM. Meanwhile, (Z)-3,4,5-trimethoxy-4′-methoxy-3′-aminosulfamatestilbene and (Z)-3,4,5-trimethoxy-4′-ethoxy-3′-aminosulfamatestilbene exhibit significant cytotoxic activity on human gastric cancer cells MKN45 and human gastric cancer cells MGC80, and the IC50 value are below 0.6 μM. In general, (Z)-3,4,5-trimethoxy-4′-ethoxy-3′-aminosulfamatestilbene has a higher positive effect on human lung cancer cell A549, human colon cancer cell HCT-116, Human cervical cancer cells HeLa, human liver cancer cells HepG2, human gastric cancer cells MGC-803, human gastric cancer cells MKN45 and other cells have significant cytotoxic activity, and the IC50 values are below 0.5 μM.

TABLE 1
Evaluation of in vitro anti-tumor activity of the compounds
against various tumor cell lines (CKK-8 method)

| Number- | | IC50 (μmol/L) | | | | |
|---|---|---|---|---|---|---|
| ing | Hela | A549 | HTC116 | HepG2 | MKN45 | MGC803 |
| CA-4 | 11.33 | 5.991 | 9.001 | 16.04 | 3.442 | 1.757 |
| Erianin | 0.2071 | 0.2199 | 6.287 | 0.2369 | 0.4255 | 0.4494 |
| A1-1 | 0.4576 | 0.4814 | 0.2216 | 0.335 | 0.5993 | 0.2977 |
| A1-2 | 4.499 | 11.39 | 1.572 | 0.8699 | 10.52 | 2.982 |
| A1-3 | 9.035 | 15.76 | 3.085 | 1.141 | 11.87 | 3.614 |
| A1-4 | 0.3166 | 0.2689 | 16.84 | 0.2625 | 0.3688 | 5.046 |
| A1-5 | 14.26 | 0.9871 | 0.3662 | 9.963 | 7.144 | 8.948 |
| A2-1 | 0.4799 | 0.2289 | 0.446 | 0.4125 | 0.3246 | 0.2674 |
| A2-2 | 5.638 | 4.858 | 9.119 | 2.631 | 8.053 | 7.633 |
| A2-3 | 2.93 | 1.604 | 10.16 | 3.647 | 7.67 | 3.586 |
| A3-1 | 7.817 | 5.21 | 9.107 | 4.805 | 9 | 12.52 |
| A3-2 | 8.419 | 28.22 | 12.84 | 5.537 | 9.242 | 13.84 |
| A3-3 | 35.65 | N.T. b | 26.48 | 15.88 | 23.26 | 71.38 |
| A4-1 | 16.3 | 10.96 | 9.499 | 9.359 | 8.534 | 15.32 |
| A4-2 | 19 | 21.53 | 40.3 | 11.85 | 27.03 | 25.04 |
| A4-3 | 27.06 | 25.83 | 52.49 | 12.03 | 17.64 | 25.7 |
| A5-1 | 0.2724 | 0.2994 | 3.286 | 0.7517 | 0.4999 | 9.564 |
| A5-2 | 0.38 | 7.421 | 8.645 | 7.531 | 6.193 | 18.87 |
| A5-3 | 2.926 | 11.95 | 30.63 | 8.103 | 8.209 | 73.88 |

### Example 27 Arylsulfatase Activity Assay In Vitro

### 1. Test Method

Weigh an appropriate amount of the test compound and dissolve it in dimethyl sulfoxide. Dilute it with Tris-HCl buffer for 2, 5, 10, 20, 50, 100, 200, 500, 1000, 2000, 5000, and 10,000 times. Add another dilution gradient to the sample. Arylsulfatase and potassium p-nitrophenyl sulfate are directly dissolved in Tris-HCl buffer. 40 μL of the test compound, 50 μL of arylsulfatase and 50 μL of potassium p-nitrobenzene sulfate were added to a 96-well plate for reaction. A blank control without enzymes and a standard control without test compounds are used. The volume is made up with Tris-HCl buffer. 405 nm detection in the microplate reader, each sample is measured for 40 times, and the interval is 30 s. The representative time node within the time period with good linearity of the absorbance value are selected for data analysis. The measured absorbance value are subtracted from the background value of the blank control and the initial value when the reaction time are 0. For comparison, calculate the inhibition rate at different concentrations, and use the built-in forecast function of excel to predict the IC50 value.

### 2. Test Results

The experimental results show that the target compound and the positive control EMATE have different degrees of sulfatase inhibitory activity. Among them, the activity of (Z)-3,4,5-trimethoxy-4′-methoxy-3′-aminosulfamate-stilbene 6.1601 μmol/mL is greater than (E)-3,4,5-trimethoxy-4′-methoxy-3′-aminosulfamatestilbene has an activity of 12.8568 μmol/mL, and it is concluded that the activity of the cis isomers are relatively better than that of the trans isomers. In addition, (Z)-3,4,5-trimethoxy-3′-sulfamatestilbene is a strong inhibitor with the activity of 0.47111 μmol/mL.

TABLE 2
Evaluation of in vitro inhibitory activity
of the compounds on sulfatase

| Number | Emate | A1-1 | A1-2 | A3-1 | A4-1 |
|---|---|---|---|---|---|
| IC50 (uM) | 5.01 | 6.16 | 12.86 | 4.64 | 0.47 |

### Example 28 Tubulin Polymerization Activity Test

### 1. Test Method

Tubulin (4.8 mg/ml) protein was mixed with the test compound in a PEM buffer (100 mM PIPES, 1 mM MgCl2 and 1 mM EGTA) containing 1 mM GTP and 5% glycerol. A SPECTRA MAX 190 (Molecular Device) spectrophotometer was used to monitor the microtubule polymerization at 37° C. by light scattering at 340 nm, and the microtubule polymerization was calculated from the absorbance value. Each sample was tested three times at ten different concentrations. The inhibition rate was: inhibition %={1 (ODsample ODblank)/(ODcontrol ODblank)}×100%, and IC50 value was determined using GAPAPD PrISM software (inhibition cell concentration was 50%).

### Test Results

TABLE 3
Evaluation of in-vitro inhibitory activity
of the compounds on sulfatase

| Compound No. | IC₅₀(uM) | SD |
|---|---|---|
| A1-1 | 6.6 | 0.8 |
| A1-2 | 55.7 | 6.0 |
| A1-3 | 72.6 | 10.7 |
| A1-5 | 3.1 | 1.1 |
| A3-1 | 1.8 | 0.0 |
| A3-2 | 12.5 | 1.5 |
| A3-3 | 86.2 | 6.4 |
| EMATE | 25.9 | 7.1 |
| CA-4 | 1.0 | 0.2 |

### Example 29 (Experiment of Tumor Inhibition Rate of Oral Administration on Sarcoma S180 Mouse Transplantation Tumor)

### 1. Test Method

After 1 week of adaptation, the mice were inoculated subcutaneously with sarcoma S180 tumor tissue. After the tumor grew 100-300 mm3, the animals were randomly divided into groups. Each compound in the medication group had 6 rats each, and the control group had 12 rats. −1 and A2-1, the doses are 25, 50 mg/kg, and the positive control is CA-4, the administration time is d0, d2, d4, d6, d8, d10, d12 days, a total of 7 times a week Measure tumor volume three times, weigh the rats, record the data, sacrifice on day 14 after inoculation, weigh the tumor mass, calculate the tumor suppression rate, tumor weight inhibition rate %=(1−average tumor weight in the treatment group/average tumor in the control group Heavy)×100%.

### 2. Test Results

According to the dosing schedule, the above compounds all significantly inhibited the growth of sarcoma S180 mice transplanted tumors. On the 8th day after administration, it is observed that the tumors in the A1-1 and A2-1 administration groups have a shrinking trend. The tumor inhibition rate was over 60% in 50 (mg/kg) group.

Tumor inhibition rate of intravenous injection of test drugs on sarcoma 5180 mice xenograft

| | | |
|---|---|---|
| Group | A1-1 | A2-1 |

| | | | | |
|---|---|---|---|---|
| dose (mg/kg) | 25 | 50 | 25 | 50 |
| Inhibition rate (%) | 42 | 70 | 40 | 65 |

### Example 30 (Acute Toxicity Test of Single Intragastric Administration of Mice)

### 1. Experimental Methods

Kunming mice (body weight: 17-22 g, male and female), randomly divided according to body weight. During the experiment, every 10 mice were used as a dose group, the highest dose was 150 mg/kg, and 0.9 was divided into 10 dose groups. The doses were 150, 130, 120, 110, 100, 90, 80, 70, 60, 50, 40, 30 mg/kg by single intragastric administration, 0.25 h, 0.5 h, 1 h, 2 h after administration Observe once every 4 h, 24 h, and then record the mortality, then observe it once a day, record the mortality for 14 days, and sacrifice the undead mice on the 15th day for pathological dissection.

### 2. Experimental Results

Single oral gavage, 40 min-1 hr of high-dose injection, the death with no obvious residual drug solution on anatomy, indicate rapid drug absorption, the rest mainly die on the first 1-2 days after administration. After a few days, no mouse death is observed. The dead mice are dissected without abnormal organs such as heart, lung, liver, spleen, and kidney. Surviving mice showed diarrhea, but not seriously, indicating that the tested drugs were mainly acute toxicity reactions. Significant delayed toxicity.

| | A1-1 | A2-1 |
|---|---|---|
| LD50 (mg/kg) | 105.6 | 101.2 |
| 95% Confidence limit | 81.5-139.2 | 78.7-129.6 |

The above are merely preferred embodiments of the present invention, and not intended to limit the scope of the essential technical content of the present invention. The essential technical content of the present invention is broadly defined in the scope of the claims of the application, and is a technical entity completed by any other or the method, if it is exactly the same or any equivalent change as that defined in the scope of claims of the application will be considered to be covered by the scope of the claims.

What is claimed is:

1. The CA-4 antitumour drug characterized in the structure is shown in Formula I: 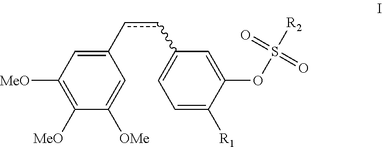
Where: “---” is a bond or does not exist.
R₁ is OMe, OEt, OCF₂H or H; R₂ is NH₂, NHMe or N(CH₃)₂.

2. A method for synthesizing the CA-4 antitumour drug according to claim 1, wherein the specific steps are as follows:
(1) The compounds having the structure shown in Formula 02 is obtained by the compounds having the structure shown in Formula 01 reacting with chlorotriphenylmethane. 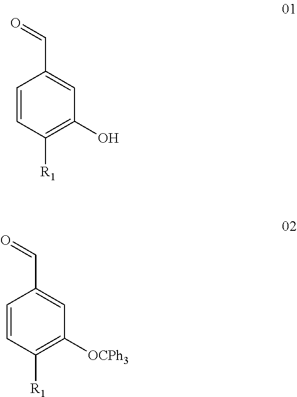
(2) The compounds with structures shown in Formulas 03 and 04 are prepared from Witting reaction of 3,4,5-trimethoxybenzyl triphenylphosphonium bromide and formula 02 using n-butyl lithium. 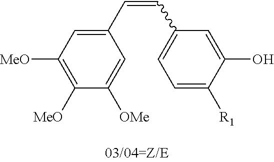
(3) The compounds of the structure shown by Formula I are obtained by reacting the sulfamoyl chlorides with the compound of the structure shown by Formula 03 and Formula 04 under a basic condition. 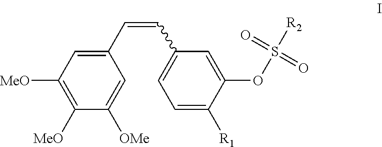
Where: “---” is a bond or does not exist.
R₁ is OMe, OEt, OCF₂H or H; R₂ is NH₂, NHMe or N(CH₃)₂.

3. The CA-4 antitumour drug, characterized in the structure are shown in Formula II: 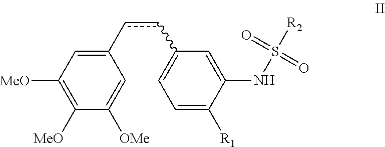
Where: “---” is a bond or does not exist.
R₁ is OMe, OEt, OCF₂H or H; R₂ is NH₂, NHMe or N(CH₃)₂.

4. The CA-4 antitumour drug, characterized in that its structure are shown by the general formula IV: 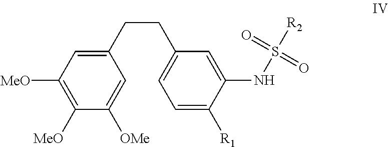
Where: “---” is a bond or does not exist.
R₁ is OMe, OEt, OCF₂H or H; R₂ is NH₂, NHMe or N(CH₃)₂.

5. A method for synthesizing the CA-4 antitumour drug according to claim 4, wherein the specific steps are as follows:
(1) The compounds with structures shown in Formulas 06 and 07 are prepared from Witting reaction of 3,4,5-trimethoxybenzyl triphenylphosphonium bromide and formula 05 using n-butyl lithium. 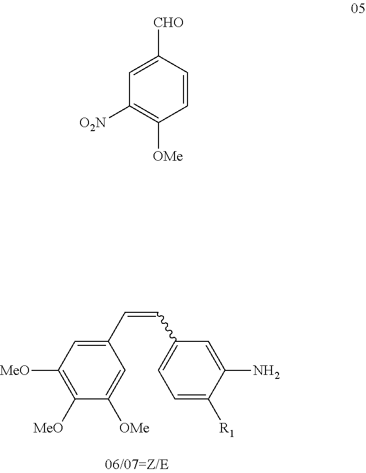
(2) The compounds of the formula III are prepared by reacting the compounds of the formulas 06 and 07 with sulfamoyl chlorides under basic condition. 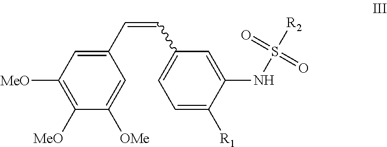
(3) The compounds with formula IV are obtained from the reduction of the compounds (formula III) using palladium/carbon catalysts. 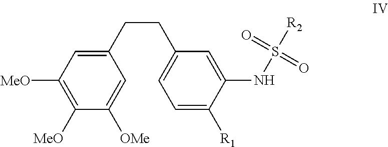
Where:
R₁ is OMe, OEt, OCF₂H or H; R₂ is NH₂, NHMe or N(CH₃)₂

6. The applications of the CA-4 antitumour drug according to claim 1 or 3 or 4 in the preparation of a tubulin aggregation inhibitor.

7. The applications of the CA-4 antitumour drug according to claim 1 or 3 or 4 in the preparation of a medicament that act as an anti-tumor vascular disrupting agent and have a vascular targeting effect on various tumors; wherein: The tumors are cervical cancer, colon cancer, lung cancer, liver cancer, breast cancer or gastric cancer.

8. The applications of the CA-4 antitumour drug according to claim 1 or 3 or 4 in the preparation of a medicament for treating a disease caused by abnormal neovascularization.
